(19) United States
(12) Patent Application Publication
(10) Pub. No.: US 2026/0077029 A1
(43) Pub. Date: Mar. 19, 2026

(54) Immunogenic Compositions of PEGylated Polysaccharide Compounds

(71) Applicant: Inventprise, Inc., Redmond, WA (US)

(72) Inventors: Subhash V. Kapre, Redmond, WA (US); Anup K. Datta, Renton, WA (US)

(73) Assignee: Inventprise, Inc., Redmond, WA (US)

(21) Appl. No.: 19/334,570

(22) Filed: Sep. 19, 2025

Related U.S. Application Data
(60) Provisional application No. 63/696,773, filed on Sep. 19, 2024.

(51) Int. Cl. A61K 39/09; A61K 39/00

(52) U.S. Cl. CPC A61K 39/092; A61K 2039/55505; A61K 2039/6093; A61K 2039/70

(57) ABSTRACT

The present invention is directed to complexes comprising multivalent compounds, immunogenic compositions, and vaccines comprising carrier protein coupled to pegylated bacterial capsular polysaccharides and uses thereof. In particular, compositions of the invention comprise bacterial capsular conjugated to polyethylene glycol (PEG) and like compounds to which are couple linkers and/or carrier proteins. PEGylated polysaccharides are derived from many different bacterial serotypes such as Streptococcus pneumoniae. The carrier protein may be coupled to the pegylated polysaccharide through homo or hetero mono-functional, bi-functional, and/or multi-functional linkers.

### REFERENCE TO RELATED APPLICATIONS

This application claims priority to U.S. Provisional Application No. 63/696,773 filed Sep. 19, 2024, the entirety of which is incorporated herein.

### BACKGROUND

### 1. Field of the Invention

The present invention is directed to complexes comprising multivalent compounds, immunogenic compositions, and vaccines comprising carrier protein coupled to pegylated bacterial capsular polysaccharides and uses thereof. In particular, compositions of the invention comprise bacterial capsular conjugated to polyethylene glycol (PEG) and like compounds to which are couple linkers and/or carrier proteins. PEGylated polysaccharides are derived from many different bacterial serotypes such as Streptococcus pneumoniae. The carrier protein may be coupled to the pegylated polysaccharide through homo or hetero mono-functional, bi-functional, and/or multi-functional linkers.

### Description of the Background

Streptococcus pneumoniae is a Gram-positive pathogen responsible for invasive pneumococcal diseases (IPDs) such as pneumonia, bacteremia, meningitis, and acute Otitis media. Pneumonia is the most common manifestation of invasive pneumococcal disease, whereas bacterial spread within the respiratory tract may result in middle-ear infection, sinusitis or recurrent bronchitis. Pneumococcus is encapsulated with a chemically linked polysaccharide which results in serotype specificity. At least 90 pneumococcal serotypes are known of which about 23 account for 90% of invasive diseases and capsular polysaccharide is a poor immunogen.

There are currently three PCV vaccines available on the global market: PREVNAR®, SYNFLORIX®, and PREVNAR-13®. There is a need to address remaining unmet medical need for coverage of pneumococcal disease due to serotypes not found in PREVNAR-13® and potential for serotype replacement over time. here is a need for immunogenic compositions covering pathogenic serotypes and methodology that can be used to induce a uniform and high immune response against all serotypes including the additional Streptococcus pneumoniae serotypes in humans and in children less than two years old.

A capsular polysaccharide (CPS) is a key virulence determinant and generally insufficiently immunogenic to induce a T cell-dependent immune response in infants and children. Most all bacterial polysaccharide vaccines are conjugations of bacterial polysaccharides with protein carrier proteins. Conjugation of a carrier protein to CPS can induce an immune response that undergoes class switching. Polysaccharide—protein conjugates tend to may have better solubility, greater stability, prolonged circulation time, and a reduced immunogenicity to the protein portion with a greater immunogenicity to the polysaccharide portion as compared to the polysaccharide portion alone. Accordingly, a 7-valent (PCV-7, Pfizer Inc., USA), a 10-valent (Synflorox-10, GSK Vaccines) and a 13-valent pneumococcal conjugate vaccine (PCV-13, Pfizer Inc., USA) have been developed to efficiently prevent the incidence of IPDs. Reductive amination chemistry and cyanylation chemistry has been widely used to prepare the conjugate vaccines.

U.S. Pat. No. 9,492,559 discloses immunogenic compositions comprising conjugated capsular polysaccharide antigens and uses thereof. The immunogenic compositions disclosed include an 8, 9, 10, 11, 12, 13, 14, 15, 16, 17, 18, 19 or 20-valent pneumococcal conjugate composition. Also disclosed is a 7, 8, 9, 10, 11, 12, 13, 14, 15, 16, 17, 18, 19, 20, 21, 22, 23, 24 or 25-valent pneumococcal conjugate composition.

International Application Publication No. WO 2014/097099A2 discloses a glycol-conjugation process directed to several serotypes in addition to Preevnar-13 valent conjugates. New polysaccharide conjugates are added to formulation to increase efficacy of the vaccine.

U.S. Patent Application Publication No. 2011/023526 discloses a 15-valent pneumococcal polysaccharide-protein conjugate vaccine composition. This patent is directed to 15-valent conjugate vaccines made by adding two or more serotypes with currently available 1-3 vaccines.

International Application Publication No. WO 2016/207905 discloses multivalent pneumococcal conjugate vaccine. This application is directed to a 13 or greater valent conjugate vaccine and deletion of serotype 6A.

U.S. Patent Application Publication No. 2017/007713 discloses a linker containing ((2-oxoethyl) thio) with enhanced functionality.

International Application Publication No. WO 2014/092377 discloses a 13 valent composition wherein 12 serotypes were selected from the group consisting of serotypes 1, 3, 4, 5, 6A, 6B, 7F, 9V, 14, 18C, 19A, 19F, and 23F and one from 12 or 9N.

International Application Publication No. WO 2014/092378 discloses an immunogenic composition having 13 different polysaccharide-protein conjugates wherein each conjugate contained a capsular polysaccharide isolated from 12 serotypes selected from the group consisting of serotypes 1,3,4,5, 6A, 6B, 7F, 9V, 14, 18C, 19A, 19F and 23F, and serotypes 22F or 33F.

Chinese Application Publication No. 101590224 discloses a 14-valent pneumococcal polysaccharide-protein conjugate vaccine containing serotypes 1, 2, 4, 5, 6A, 6B, 7F, 9N, 9V, 14, 18C, 19A, 19F and 23F.

Chinese Application Publication No. 104069488 discloses 14 valent polysaccharide protein conjugates wherein the 14 serotypes were 1,4,5, 6A, 6B, 7F, 9V, 14, 18C, 19A, 19F, 22F, 23F and 33F.

International Application Publication No. WO 2016207905 discloses a multivalent pneumococcal conjugate vaccine comprising conjugates of CRM197 and at least 14 capsular polysaccharides selected from serotypes 1, 3, 4, 5, 6B, 7F, 9N, 9V, 14, 15B, 18C, 19A, 19F, 22F, 23F and 33F. U.S. Pat. No. 8,192,746 disclosed a 15 valent immunogenic composition comprising capsular polysaccharides from serotypes 1,3,4,5, 6A, 6B, 7F, 9V, 14, 18C, 19A, 19F,22F, 23F, and 33F conjugated to CRM197.

International Application Publication No. WO 2013/191459 discloses a 15 valent composition comprising S. pneumoniae capsular polysaccharides form serotypes of 1,2,3,4,5, 6A, 6B, 7F, 9N, 9V, 14, 18C, 19A, 19F and 23F.

Chinese Application Publication No. 103656632 discloses multi valent pneumococcal capsular polysaccharide composition containing serotype 6A and at least one extra serotype selected from the group consisting of 1, 2, 3, 4, 5, 6B, 7F, 8, 9N, 10A, 11A, 12F, 14, 15B, 17F, 18C, 19A, 19F, 20, 22F, 23F and 33F which provided protection against 24 different pneumococci serotypes.

Chinese Application Publication No. 103656631 discloses a multivalent pneumococcus capsular polysaccharide-protein conjugate composition comprising capsular polysaccharides of pneumococcus of 24 different serotypes viz. 1, 2,3, 4, 5, 6A, 6B,7F, 8, 9N, 9V, 10A, 11A, 12F, 14, 15B, 17F, 18C, 19A, 19F, 20, 22F, 23F and 33F.

U.S. Patent Application Publication No. 2016/0324950 discloses immunogenic polysaccharide-protein conjugates comprising a capsular polysaccharide (CP) from Streptococcus agalactiae, also referred to as group B Streptococcus (GBS), and a carrier protein, wherein the CP is selected from the group consisting of serotypes Ia, Ib, II, III, IV, V, VI, VII, VIII, and IX. This was meant for treatment of chronic diabetes mellitus, cancer, heart failure, neurologic, and urologic conditions. The carrier protein capsular polysaccharide conjugates varied.

U.S. Pat. No. 5,360,897 discloses immunogenic conjugate comprising reductive amination product of an intact capsular polymer of the bacterial pathogen S. pneumoniae having at least two carbonyl groups and a bacterial toxin or toxoid, said conjugate comprising a cross-linked conjugate in which there is a direct covalent linkage between the capsular polymer and the toxin or toxoid.

U.S. Pat. No. 7,862,823 describes a multivalent conjugate vaccine composition with at least two different carrier proteins.

U.S. Pat. No. 8,808,708 discloses a 13-valent immunogenic composition consisting of polysaccharide-protein conjugates where serotypes consist of 1,3,4,5, 6A, 6B, 7F, 9V,14, 18C, 19A, 19F and 23F, and wherein the carrier protein is CRMI97.

U.S. Patent Application Publication No. 2009/0017059 discloses an immunogenic composition where serotypes 19A and 19F were conjugated to different bacterial toxoids.

International Application Publication No. WO 2011/110241 describes pneumococcal conjugate immunogenic compositions or vaccines wherein different conjugation chemistries were used for different components of the immunogenic composition or vaccine. Reductive amination was used for the conjugation of at least one serotype and a conjugation other than reductive amination was used for the conjugation of a different serotypes. The conjugation method selected for different serotypes allowed each serotype to be presented using a conjugation method that allowed the best presentation of the saccharide epitope. Some pneumococcal saccharides conjugated well using reductive amination, whereas other pneumococcal saccharides were conjugated differently to allow the ring structure to remain unbroken and provide better results.

U.S. Pat. No. 7,955,605 discloses a process of making carrier protein polysaccharide conjugate consisting serotype 19A where the activated serotype 19A polysaccharide and carrier protein are suspended in dimethyl sulfoxide (DMSO) to form a conjugate.

U.S. Patent Application Publication No. 2010/0074922 discloses immunogenic composition containing 10 or more serotypes wherein 19F capsular saccharide was conjugated to diphtheria toxoid (DT), serotype 18C capsular saccharide is conjugated to tetanus toxoid and serotypes 1,4,5, 6B, 7F, 9V, 14 and 23F capsular saccharides are conjugated to Protein D from Haemophilus influenza.

U.S. Patent Application Publication No. 2010/0239604 discloses a composition comprising multivalent S. pneumoniae capsular saccharide conjugates wherein serotype 19A was conjugated to a first bacterial toxoid and 19F is conjugated to a second bacterial toxoid and 2-9 of the S. pneumoniae capsular saccharides are conjugated to protein D. Apart from increasing the scope of protection by developing vaccines which will offer protection against larger number of serotypes, efforts were focused on developing newer methods of synthesis.

U.S. Pat. No. 7,709,001 describes a method of synthesis of carrier protein conjugate of capsular polysaccharide which consists of 1) reacting purified polysaccharide with a mild acid resulting in size reduction 2) reacting the polysaccharide of step 1 with an oxidizing agent in the presence of bivalent cations resulting in an activated polysaccharide; 3) compounding the activated polysaccharide with a carrier protein 4) reacting activated polysaccharide of step 3 and carrier protein with a reducing agent to form a polysaccharide carrier protein conjugate; and 5) capping unreacted aldehydes in product of step 4 to yield an immunogenic polysaccharide-carrier protein conjugate.

International Application Publication No. WO 2014/097099 discloses a method of synthesizing a carrier protein conjugate, which involves a) reacting a saccharide with 2,2,6,6-tetramethyl-1-piperidinyloxy (TEMPO) and N-chlorosuccinimide (NCS) in an aqueous solvent to produce an activated saccharide; and b) reacting the activated saccharide with a carrier protein comprising one or more amine groups.

U.S. Patent Application Publication No. 2012/321658 discloses an immunogenic composition wherein serotypes 1,3, 19A and 19F linked to protein carriers either directly or indirectly through a chemistry other than reductive amination, and one or more different saccharides is/are selected from a second group consisting of serotypes 4, 5, 6A, 6B,7F, 9V, 14, 18C and 23F which is/are linked to a protein carriers) by reductive amination.

Pneumococcal vaccines are based on 1) pneumococcal polysaccharide vaccine and 2) pneumococcal conjugate vaccines. PNEUMOVAX® marketed by Merck comprises of unconjugated polysaccharides belonging to serotypes 1,2,3,4,5, 6B, 7F, 8, 9N, 9V, 10A, 11A, 12F, 14, 15B, 17F, 18e, 19F, 19A, 20, 22F, 23F and 33F. Infants and young children respond poorly to most pneumococcal polysaccharides. Immunogenicity of poor immunogens is enhanced by conjugating with carrier proteins. Polysaccharide protein conjugate vaccines are made using capsular polysaccharides linked to protein carriers. The conjugate induces T cell dependent enhanced immune response against the specific serotype.

Conjugates are synthesized using various reagents, such as homo bifunctional, hetero bifunctional linkers of varying lengths. Three pneumococcal conjugate vaccines are available in market, PREVNAR®, SYNFLORIX®, and PREVNAR-13®. PREVNAR® is a heptavalent vaccine that contains the capsular polysaccharides from serotypes 4, 6B, 9Y, 14, 18C, 19F and 23F, each conjugated to a carrier protein designated CRM197. SYNFLORIX® is a deca-valent vaccine from GSK Biologicals that incorporates ten capsular polysaccharides conjugated to protein D from NTHi offering coverage against three additional pneumococcal strains, serotypes 1, 5 and 7F. PREVNAR-13® is a tri-deca-valent vaccine containing 13 capsular polysaccharide prepared from thirteen serotype of Streptococcus pneumoniae (1, 3, 4, 5, 6A, 6B, 7F, 9Y, 14, 18C, 19 A, 19F, and 23F) conjugated to a carrier protein designated CRM197.

Increasing microbial resistance to antibiotics and the increasing number of immunocompromised persons have necessitated the development of pneumococcal vaccines with even broader protection, which leads to development of multivalent vaccines effective against increasing number of serotypes especially for coverage of pneumococcal disease due to serotypes not found in PREVNAR-13®. The need for a specific serotype depends on the region and antibiotic resistance developed. Thus, U.S. Pat. No. 8,192,746 reports a multivalent immunogenic composition having 15 distinct polysaccharide-protein conjugates. Each conjugate consists of a capsular polysaccharide prepared from serotype of Streptococcus pneumoniae (1, 3, 4, 5, 6A, 6B, 7F, 9\1, 14, 18C, 19A, 19F, 22F, 23F, or 33F) conjugated to a carrier protein CRM197. There is a need for vaccines that induce an immune response against serotype 15B, 15C, and 15A.

With the current methods increasing number of polysaccharide antigens in the multivalent conjugate vaccine formulations, the carrier protein content increases. This increase leads to an increase of immune response to the carrier protein which can cause a systemic overload. This needs to be reduced. Also, there is a lowering of immune response as the serotypes increase, which needs to be increased.

Thus, there is a need to develop vaccine and in particular a pneumococcal vaccine that provides protection against large numbers of serotypes, with little to no immunogenicity of the carrier protein. Preferably, the immune response to individual serotypes is largely unaffected by the number of serotypes to which the vaccine may be directed. These desired multivalent vaccines should extend an immune stimulus for existing and additional serotypes. In addition to offering suitable protection against increasing number of serotypes, there is also a need to develop methods to reduce carrier protein antibodies in spite of an increase in the number of serotypes.

### SUMMARY OF THE INVENTION

The present invention overcomes the problems and disadvantages associated with current strategies and designs and provides new compositions and methods creating uniform high immune response that decrease in the antibody response to the carrier protein with an unlimited number of serotypes. This is done by conjugating a polysaccharide with PEG before coupling with a carrier protein. The PEG molecules chosen are preferably below 1 KDa in molecular weight to avoid any PEG antibody generation.

One embodiment of the invention is directed to immunogenic complexes comprising a multivalent capsular polysaccharides conjugated to PEG. Thereafter, the conjugated molecules may be coupled to a carrier protein. The number of different bacterial serotypes that can be formed into the immunogenic complex may be from 1 to 100 or more. Preferred S. pneumoniae serotypes include, for example, serotypes 1, 2, 3,4, 5, 6A, 6B, 6C, 6D,7F, 8, 9V, 9N, 9A, 9B,10A, 11A, 12F, 14, 15B, 15A, 15B, 15C, 17F, 18C, 19A, 19B, 19F, 19N, 19V, 20, 22F, 23F, 24F, 33F and 35B. The multivalent S. pneumoniae complexes comprises groups of conjugated compounds which may be coupled to one or more linkers and/or one or more carrier proteins. Coupling multiple conjugated PEFG-PSs reduces the quantity of carrier protein needed and, consequently, reduces the risk of an unwanted immunological response to carrier protein. Complexes of the disclosure involve one carrier protein molecule for multiple pegylated-polysaccharides. As this mechanism provides a precise attachment to a specific amino acid group in the protein, the method results in a consistent coupling with same ratios of polysaccharides to protein. Thus, one can use diverse complexes of pegylated polysaccharides and reduce antibody quantity per dose, lowering the immune response to carrier protein, but keeping the polysaccharide response equivalently or higher. For example, conjugating two micrograms of one serotype and also two micrograms of another polysaccharide would involve half the dose reducing both the polysaccharide quantity as well as carrier protein quantity without any reduction in the protective response.

This methodology is effective for cross reactive serotypes wherein reduction in quantity in a single dose would not reduce the total immune response which would happen if conventional patented and published methods were to be used. Preferably, complexes of the disclosure contain conjugated forms of PEG-PSs of variety of different bacterial serotypes including serotypes of different types or species of bacteria. In other words, the disclosure includes multi-functional complexes using antigens from different bacterial sources not belonging to Pneumo group to avoid making another vaccine for immunization, such as compounding HIA polysaccharide and HIB polysaccharide to a common carrier protein.

Preferably, the complex comprises PEG conjugated capsular polysaccharide of two immunologically cross-reactive serotypes coupled to the same carrier protein sequentially or concurrently. Preferably bacterial capsular polysaccharides have molecular weight ranges of about 10 KDa to about 50 KDa, about 30 KDa to about 100 KDa, or about 100 KDa to about 300 KDa.

Preferably, the PEG-capsular polysaccharide of two immunologically cross-reactive serotypes is represented, for example, by the formula PEG-PS1; PEG-PS2; PS1-PEG-PS2; and similar permutations. Preferably the complexes are coupled to a carrier protein and preferred carrier proteins include, but are not limited to Tetanus Toxoid, Diphtheria Toxoid, CRM197, Tetanus Toxoid fragments (TTHc), N. meningitidis protein PorB, RSV virus proteins, B. pertussis proteins, Pertussis toxoid (PT), Adenylate cyclase Toxin (ACT), 69 KDa protein, Human Papilloma viral protein antigens, Human Papilloma virus VLP forms, Hepatitis B virus core antigen, Hepatitis B virus VLP forms, derivatives of HBsAg, or combinations thereof. Preferably a single dose of bivalent cross-reactive polysaccharide compound comprises less than 4 micrograms in comparison to monovalent compounds of the same two polysaccharide vaccines which would add up to about 4 micrograms or more.

Preferably, total carrier protein quantity in the multivalent PEG-PS complexes is significantly lower than the quantity used with conventional compounds of the individual polysaccharides coupled to carrier protein. Preferably, the vaccines of the present invention, the carrier protein amount being compounded to a multivalent cross-reactive PEG conjugated polysaccharide has less protein per serotype in comparison to that of the monovalent compounds of two polysaccharides to one protein thereby reduce the carrier protein immune response generated by the latter vaccine compound Preferably total carrier protein content in the multivalent compounded vaccine is from 0.5 to about 0.7 % by weight of the mono compounds of the individual polysaccharides of the same cross-reactive serotypes (which is 1:1 ratio between PEG-PS: Carrier Protein).

Preferably, the vaccine further comprises at least one adjuvant selected from the group consisting of aluminum or an aluminum salt, calcium phosphate, a liposome of monophosphoryl lipid A (MPLA), saponin QS-21, and/or a potent TLR7/8 agonist. Preferably the at least one adjuvant comprises an aluminum adjuvant selected from the group consisting of aluminum phosphate, aluminum sulfate and aluminum hydroxide.

Preferably the bacterial polysaccharides are selected from the group consisting of cross reacting two or more serotypes from different bacterial capsular polysaccharides and/or the bacterial polysaccharides comprise: S. pneumoniae and H. influenza type a, b serotypes; S. pneumoniae and Group B Streptococcus serotypes, H. influenza type a, b serotypes, or N. meningitis serotypes. Preferably the capsular polysaccharides of PEG-PS complexes comprise polysaccharides derived from Streptococcus pneumoniae, Haemophilus influenza, N. meningitis, Group B Streptococcus, Klebsiella pneumoniae, and/or Moraxella catarrhalis lipo-oligosaccharides (LOS). Preferably, the capsular polysaccharide is derived from Haemophilus influenza serotypes a/b/c/d/e/f, non-typeable Haemophilus influenza (NTHi) polysaccharides, or Moraxella catarrhalis Lipooligosaccharides (LOS), or N. meningitis serotypes A, B, C, Y, W-135 or X; Group B Streptococcus serotypes Ia, Ib, II, III, IV, V, VI, VII, VIII. IX and N; N. meningitis serotypes A, C, Y, X, and W-135; and/or on or more of serologically defined sub-capsular O types and the 77 capsular K types of Klebsiella pneumoniae.

Another embodiment of the invention is directed to immunologically active PEG-PS complexes and vaccines for the treatment or prevention of infection by Gram-positive and Gram-negative pathogens comprising a therapeutically effective amount of the complex vaccine of the invention and, optionally, a pharmacologically acceptable carrier. Preferably the capsular polysaccharides are derived from Haemophilus influenza, N. meningitis, Group B Streptococcus, N. meningitis, H. influenza, Klebsiella pneumoniae, Moraxella catarrhalis lipo-oligosaccharides (LOS), and combination thereof.

Another embodiment of the invention is directed to methods for conjugating polysaccharides to PEG; after which the PEG conjugated PSs are activated; attaching to spacers, linkers, and/or carrier proteins. A defined length of a spacer arm of about 2.0 Å to about 40 Å to the activated PEG-conjugated polysaccharide; and attaching the activated PEG-conjugated polysaccharide attached to a spacer arm to a carrier protein. PEG acts as an unexpected booster to immune response to polysaccharides which also acts to reduce the response to the carrier protein. The result is a highly effective immunogenic complex with reduced detrimental effects.

Another embodiment of the invention is directed to methods coupling the PEG-conjugated PSs to one or more carrier proteins. Preferably, multiple PEG-conjugated PSs are coupled to a single carrier protein. For example, PEG-conjugated PSs are activated and the activated molecules couple to carrier proteins. Preferably the carrier proteins disulfide bonds are reduced to create sulfhydryl groups creating a sulfhydryl group using 2-iminothiolane (2-IT). Carrier molecules or PEG-conjugated PSs may be coupled to linkers or spacers, such as SMPH like bi-functional, tri-functional or multi-functional PEG linker; attaching a defined length PEG spacer arm of about 4 Å to about 40 Å to the activated carrier protein; and then attaching the polysaccharide to a similar PEG spacer arm attached to activated carrier protein. Preferably the activated carrier protein is selected from cross-reactive material (CRM197) obtained or derived from C. diphtheria, or recombinant CRM197 obtained or derived from P. fluorescens or E. coli.

Another embodiment of the invention is directed to immunogenic complexes containing PEG-conjugated PSs that are homo- or hetero-multifunctional, such as combinations of different genus of bacterial polysaccharides as well as another species and/or genus of PEG-conjugated bacterial polysaccharides.

Another embodiment of the invention is directed to multivalent S. pneumoniae vaccine compound wherein carrier protein is cross-reactive material (CRM197) obtained from C. diphtheria, recombinant CRM197 obtained from P. fluorescens, or recombinant CRM197 obtained from E. coli.

Other embodiments and advantages of the invention are set forth in part in the description, which follows, and in part, may be obvious from this description, or may be learned from the practice of the invention.

### DESCRIPTION OF THE INVENTION

Streptococcus pneumoniae is a Gram-positive bacterium which can cause diseases such as pneumonia, bacteremia, meningitis, and acute Otitis media. Pneumococcus is encapsulated with a chemically linked polysaccharide which results in serotype specificity. At least 90 pneumococcal serotypes are known of which about 23 account for 90% of invasive diseases. The protection against invasive pneumococci disease is related to the antibody specific to the capsular polysaccharide, the protection is therefore serotype specific.

It was surprisingly discovered that multivalent vaccines comprising of polysaccharide conjugated to PEG before coupling to carrier protein provides for a stable molecule that allows for the formation of multivalent immunogenic complexes and ultimately vaccines. Results achieved are enormously improved compared to vaccines containing multivalent vaccines without PEG-conjugated PSs.

The present disclosure is directed to multivalent PEGylated polysaccharides forming immunogenic compositions, and vaccines. Preferably, the complexes form multivalent immunogenic compositions. Preferred multivalents complexes and compositions comprise 25 or more different PSs, 30 or more different PSs, 35 or more different PSs, 40 or more different PSs, 45 or more different PSs, 50 or more different PSs, 55 or more different PSs, 60 or more different PSs, 65 or more different PSs, 70 or more different PSs, 75 or more different PSs, 80 or more different PSs, 85 or more different PSs, 90 or more different PSs, 95 or more different PSs or 100 or more different PSs. PSs may be some the same or different microorganisms including, but not limited to species, types and/or serotypes of Streptococcus, Pneumococcus, Group A and/or B Streptococcus, Bacillus, Nisceria, Haemophilus, non-typeable Haemophilus, Moraxella, Mycobacteria, Meningococcus, Clostridium, Salmonella, Escherichia, Listeria, Campylobacter, Corynebacterium, Bordetella, **. Complexes can be coupled, preferably covalently, with carrier protein. Complexes and carrier proteins can be coupled via spacer and/or linker such as PEG-linkers. Preferably, the PEGylate polysaccharide is connected to a carrier protein via two hydrazine functional groups cable of covalently compounding with both carrier protein as well as the PEGylated polysaccharides. This creates a new class of covalently compounded PEG products that have the additional effect of PEG on their properties compared to conjugates made by established methods. PEG has an additional enhancing effect on the immunogenicity of polysaccharides compared to regular conjugates and a depressing effect on the Immune response of carrier proteins. As the compounds of the invention contain PEGylated polysaccharide associated with and/or coupled to carrier, there is no conjugation between polysaccharide and protein.

This disclosure provides a universal method for the PEGylation of multiple polysaccharides forming complexes which possess high immune responses, eac of which is significantly unaltered individually in spite of increase in serotypes. This unexpected beneficial observation is critically important in developing immunogenic multivalent vaccines.

Protection against pneumococcal disease is obtained by antibodies produced against the polysaccharide component. By PEGylation, the response observed is twice that of PREVNAR®. This means that the high antibodies observed after administration of the PEGylated form of the polysaccharide vaccine will fall slowly, much more slowly that the rapid reductions observed with PREVNAR®. This is an entirely unexpected and extremely beneficial outcome. This result eliminates any need of multiple inoculations saving cost as well as pain to infants and others caused by multiple injections, and in addition, makes protection more widely available, especially for those unable to return for repeated injections.

In particular, compositions of the invention comprise any number of bacterial capsular polysaccharides covalently connected to same protein to form PEGylated complexes of PEG-polysaccharides of various serotype antigens wherein the bacterial capsular polysaccharides and oligosaccharides are derived from serotypes of Streptococcus pneumoniae. The carrier protein, preferable, is covalently connected to bacterial capsular polysaccharides through multi-functional spacer and/or lnkers which may also be PEGylated. Preferably these liners/spacers have defined lengths and may be homo- or hetero-functional (subgroup Serotypes like 6A-6B or 19A-19F, and/or all other serotypes).

One embodiment of the invention is directed to multivalent covalently connected complexes and vaccines comprised of multivalent-PEGylated polysaccharides with enhanced immunogenicity. Complexes may have the general structure PEG-PS1-carrier protein-PEG-PS2, which have higher immunogenicity compared to similar monovalent conjugates wherein PS1 and PS2 are non-PEGylated. By developing multivalent PEGylated polysaccharides as vaccine, the efficacy of the vaccine increases and carrier immunogenicity is reduced. The chemistry disclosed herein substantially increases immunogenicity, at the same time reduces carrier protein load.

Another embodiment of the invention is directed to vaccines with lower molecular weight polysaccharides and longer arm multifunctional linkers preferably with enhanced immunogenicity. Another embodiment of the invention is directed to providing higher immunogenicity and avidity of multivalent complexes as well as lower carrier protein immunogenicity. Another embodiment of the invention is directed to reducing covalently compounded vaccine dose with higher immunogenicity.

As disclosed herein, four parameters have been introduced to minimize the disadvantages of conventional vaccines:
Polysaccharides are conjugated to PEG.
Polysaccharide size is preferably 10-50 KDa.
Cross-reactive polysaccharides concurrent covalent connection to carrier protein.
Two or more cross reactive serotypes are covalently compounded concurrently with carrier proteins.
A long hetero-or homo-functional spacer arm is preferably from 2-40Å (but may be from 2-40 Å, 4-40 Å, 10-40 Å, 20-40 Å, 9-20 Å, 5-20 Å, 5-30 Å).

These five parameters taken together are profoundly effective to increase the polysaccharide/protein ratio in the covalent compound to reduce carrier protein load, and to provide several folds of increase in immunogenicity and avidity.

The present invention is directed to PEGylated polysaccharide complexes, which may be associated with or coupled to a carrier protein, with enhanced immunogenicity displaying significantly high antibody titers. The carrier protein is obtained from, for example, tetanus toxoid, diphtheria toxoid, CRM197, tetanus toxoid fragments (TTHc), Nisceria meningitidis protein PorB, RSV virus proteins, Bordetella pertussis proteins like pertussis toxoid (PT), adenylate cyclase toxin (ACT), 69 KDa protein and Human Papilloma viral protein antigens or its VLP form, Hepatitis B core antigen or its VLP form or derivatives of HBsAg, and other conventional carriers. Polysaccharide fragments are obtained from gram positive bacteria and/or gram-negative bacteria, preferably from immunochemically cross-reactive polysaccharides of S. pneumoniae. The present invention is also directed to a process of preparing the PEGylated polysaccharide with PEGylated carrier protein in which carrier protein reacts with cleaved and depolymerized polysaccharide fragments of optimum chain length.

Immunogenic compositions of the present invention provide improved protection against S. pneumoniae serotypes not found in PREVNAR-13®, and SYNFLORIX-10®.

Multivalent complexes with cross-reactive polysaccharides multiple serotypes of S. pneumoniae serotypes (e.g., serotypes 1, 2, 3,4, 5, 6A, 6B, 6C, 6D,7F, 8, 9V, 9N, 9A, 9B,10A, 11A, 12F, 14, 15B, 15A, 15B, 15C, 17F, 18C, 19A, 19B, 19F, 19N, 19V, 20, 22F, 23F, 24F, 33F and 35B, and similar cross-reactive serotypes) are prepared with a valency of upwards of 20 different serotypes, upwards of 25 different serotypes, upwards of 30 different serotypes, upwards of 35 different serotypes, upwards of 40 different serotypes, upwards of 45 different serotypes, upwards of 50 different serotypes, upwards of 55 different serotypes, upwards of 60 different serotypes, upwards of 65 different serotypes, upwards of 70 different serotypes, upwards of 75 different serotypes, and upwards of 80 different serotypes. Preferably CRM197 is the carrier protein for its clinical acceptance.

Multivalent complexes of PEGylated polysaccharide are also prepared using PS sizes of up to 50 KDa, spacer arms of 9-40 Å or longer with spacers and/or linker which may also be PEGylated.

CPS was activated either by oxidation or by cyanylation chemistry and oxidized by sodium periodate and introduced with either-reactive aldehyde or isothiocyanate (—OCN) groups in CPS. **

Two strategies (short and long linker, short and long CPSs) were used to introduce, respectively. Physicochemical and immunological characteristics of the bivalent covalent compound vaccines were then investigated independently or combining with multivalent compound formulation.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. A multivalent immunogenic complex comprising bacterial capsular polysaccharides conjugated to polyethylene glycol.

2. The complex of claim 1, wherein the multivalency comprises 25 or more different polysaccharides (PSs), 35 or more different PSs, 45 or more different PSs, 55 or more different PSs, 65 or more different PSs, 75 or more different PSs, or 85 or more different PSs.

3. The complex of claim 1, wherein the bacterial capsular polysaccharides comprise multiple serotypes of S. pneumoniae.

4. The complex of claim 1, wherein the bacterial capsular polysaccharides are derived from S. pneumoniae.

5. The complex of claim 4, wherein the bacterial capsular polysaccharides of S. pneumoniae comprise serotypes 1, 2, 3,4, 5, 6A, 6B, 6C, 6D,7F, 8, 9V, 9N, 9A, 9B, 9N, 9V, 10A, 11A, 12F, 14, 15A, 15B, 15C, 17F, 18C, 19A, 19F, 20, 22F, 23F, 24F, 33F and 35B.

6. The complex of claim 1, wherein the capsular polysaccharides have a molecule weight of from about 10 kDa to about 300 KDa.

7. The complex of claim 1, wherein the polyethylene glycol has a molecule weight of from about 1 kDa to about 50 KDa.

8. The complex of claim 1, which is associated with a carrier molecule.

9. The complex of claim 8, wherein association is via covalent coupling.

10. The complex of claim 8, wherein the carrier molecule comprises a protein.

11. The complex of claim 10, wherein the protein comprises tetanus toxoid, diphtheria toxoid, CRM197, tetanus toxoid fragments (TTHc), Nisceria meningitidis protein PorB, RSV virus proteins, Bordetella pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof.

12. The complex of claim 1, which comprises 4 micrograms or less of total polysaccharides per dose.

13. The complex of claim 8, which comprises from about 0.5% to about 0.7% by weight of bacterial capsular polysaccharide and carrier molecule per dose.

14. The complex of claim 8, which comprises about equal amount by weight of capsular polysaccharides to total carrier molecule.

15. The complex of claim 8, which comprises a greater amount by weight of capsular polysaccharides to total carrier molecule.

16. The complex of claim 1, further comprising of at least one adjuvant.

17. The complex of claim 16, wherein the adjuvant is selected from the group consisting of aluminum salt, calcium phosphate, a liposome of monophosphoryl lipid A (MPLA), saponin QS-21, a TLR7/8 agonist, and combinations thereof.

18. The complex of claim 17, wherein the aluminum salt is selected from the group consisting of aluminum phosphate, aluminum sulfate and/or aluminum hydroxide.

19. The complex of claim 1, comprising one or more serotypes of S. pneumoniae, H. influenza type a or b; S. pneumoniae, Group B Streptococcus, N. meningitis or combinations thereof.

20. The complex of claim 1, wherein the capsular polysaccharides are derived from S. pneumoniae, Group B Streptococcus serotypes Ia, Ib, II, III, IV, V, VI, VII, VIII, IX, or N, Haemophilus influenzae serotypes a/b/c/d/e/f, non-typeable Haemophilus influenzae (NTHi), Moraxella catarrhalis Lipooligosaccharides(LOS), N. meningitis serotypes A, B, C, Y, W-135 or X, or combinations thereof.

21. The complex of claim 1, which, upon administration to a subject, generates a lower immune response to carrier protein in comparison to monovalent conjugates comprised of the same capsular polysaccharides.

22. The complex of claim 1, which provides effective treatment or prevention of infection by Gram-positive and/or Gram-negative bacteria.

23. The complex of claim 1, further comprising a therapeutically effective amount and a pharmacologically acceptable carrier.

24. The method for manufacture of complexes of PEGylated polysaccharides comprising:
conjugating PEG to multiple bacterial polysaccharides forming PEGylated polysaccharides;
activating the PEGylated polysaccharides;
coupling spacer/linker molecules to the activated PEGylated polysaccharides, wherein the linker/spacer is about 2.0 Å to about 40 Å; and
coupling activated PEGylated polysaccharide to carrier proteins forming complexes.

25. The method for manufacture of a complex of PEGylated polysaccharides comprising:
activating carrier proteins to form activated carrier proteins;
reducing a disulfide of each carrier protein to create a sulfhydryl group; and
coupling PEGylated polysaccharides to the activated carrier proteins forming the complex.

26. The method of claim 25, wherein the activated carrier proteins are selected from the group consisting of cross-reactive material (CRM197) obtained or derived from C. diptheriae, and recombinant CRM197 obtained or derived from P. fluorescens or E. coli.
